(19) United States
(12) Patent Application Publication
(10) Pub. No.: US 2011/0196353 A1
(43) Pub. Date: Aug. 11, 2011

(54) LED Treatment of Dermatologic Toxicities Associated with Epidermal Growth Factor Receptor Inhibitors

(76) Inventors: Maitland M. DeLand, Lafayette, LA (US); Robert A. Weiss, Baltimore, MD (US)

(21) Appl. No.: 13/020,341

(22) Filed: Feb. 3, 2011

Related U.S. Application Data
(60) Provisional application No. 61/301,850, filed on Feb. 5, 2010.

(51) Int. Cl. A61B 18/18

(52) U.S. Cl. 606/9

(57) ABSTRACT

The present invention relates generally to methods of preventing or treating toxicities of the skin, hair, and/or nails, which are associated with administration of one or more epidermal growth factor receptor inhibitors, with light-emitting diode photomodulation treatment, either alone or in combination with other agents.

This application claims the benefit of priority to U.S. Provisional Application No. 61/301,850, filed on Feb. 5, 2010, the contents of which are hereby incorporated by reference in their entirety.

### FIELD OF INVENTION

The present invention relates generally to methods of preventing or treating toxicities of the skin, hair, and/or nails, which are associated with administration of one or more epidermal growth factor receptor inhibitors, using light-emitting diode photomodulation treatment, either alone or in combination with other agents.

### BACKGROUND OF THE INVENTION

The human epidermal growth factor receptor (EGFR) gene product, a member of the ErbB family of receptor tyrosine kinases, is an integral component of signaling in epithelial cell proliferation. Therapies for cancer which target this receptor have become an important part of the standard treatment regimen, as small molecules and monoclonal antibodies that inhibit the EGFR have shown promise in managing many different forms of cancer. Among the commonly used EGFR inhibitors in cancer therapy are cetuximab (Erbitux®), erlotinib (Tarceva®), gefitinib (Iressa®), and panitumumab (Vectibix®).

A side of effect of treatment with EGFR inhibitors is the presentation of dermatologic toxicities that can manifest on many areas of the body, and in particular on the face, cheeks, and back of patients, as well as toxicities presenting on the nails and affecting hair follicles are hair growth. The dermatologic toxicities can include acneiform rashes such as papulopustular rashes, as well as psoriasis, pruritus, paronychia, and changes in hair growth. Patients may also develop various other skin rashes, and problems relating to the eyelids and eyelashes. Hand and foot blisters can also be associated with EGFR inhibitor treatment because these agents can damage the capillary endothelia. Because hand and foot surfaces are under pressure from walking and other activity, the skin in these areas is more sensitive, and pressure points can develop to contribute to the blisters and erythema.

These dermatologic toxicities can begin to manifest soon after EGFR inhibitor treatment or up to several months following the end of treatment. In some circumstances, conditions such as psoriasis can develop after the papulopustular rash has resolved.

In the case of skin rashes, typically the rashes associated with EGFR inhibitor treatment appear within the first two to five weeks of treatment. The severity of the rash can vary throughout treatment, and can depend on the specific EGFR inhibitor used, and can even resolve, temporarily, throughout the duration of treatment. Once treatment discontinues, however, these initial dermatologic toxicities can disappear within a month. Depending on the specific drugs utilized, between about 45 and 100% of patients will develop a rash from treatment with EGFR inhibitors.

Long-term effects associated with EGFR inhibitor treatment include hypopigmentation and psoriasis, and within two to four months after treatment, paronychia and fissuring can develop and endure for several months.

Toxicities caused by EGFR inhibitors often also lead to secondary infections in many patients.

Patients describe the rashes associated with EGFR inhibitor treatment as pruritic and itching and most deal with chronic discomfort as well as with the appearance of the rash, which is frequently on areas of the body visible to the public. In addition, as a result of the rashes, studies indicate that about one third of patients that are administered EGFR inhibitors develop super-infections in addition to the initial rashes. Patients often discontinue treatment not only because of the physical pain and infections, but because the side effects may damage their self-image and self-esteem. Some studies indicate that about one-third of patients treated with EGFR inhibitors discontinue treatment due to the negative side effects. Frequently, therefore, physicians lower the EGFR inhibitor dosage to decrease the scale of the side effects, and in many cases, treatment is delayed as a result.

Importantly, there is a positive correlation between the severity of the rash and how effective an EGFR inhibitor is in treating a patient's cancer. Studies demonstrate this positive correlation between development of the rash and clinical outcomes, including tumor response, progression free survival, and overall survival. Therefore, a means of preventing and/or treating side effects that might hinder the administration of EGFR inhibitors is crucial.

Many treatments to manage EGFR inhibitor side effects on the skin and related areas have been attempted, including the use of tetracycline, mild cleansers, hydrocortisone, clindamycin gel, and tacrolimus cream, as well as sunscreen and analgesics. These agents have proved largely unsuccessful in treating the toxic side effects of EGFR inhibitor treatment, and therefore there is a need for additional therapies that are efficacious in preventing and treating these unpleasant side effects.

### SUMMARY OF INVENTION

This invention encompasses methods of treating and preventing toxicity of the skin, hair, and nails, that is associated with the administration of EGFR inhibitors, comprising light-emitting diode (LED) photomodulation therapy, either alone or in combination with other therapies. Subjects for use with this invention are mammalian, and preferably are human. By treating patients in need of treatment using the methods of this invention one can manage, attenuate, ameliorate, or prevent the progression of skin, hair, and/or nail toxicity associated with the administration of EGFR inhibitors.

LED photomodulation therapy is particularly effective for preventing and treating toxicities associated with EGFR administration in patients with different types of cancers, as well as for preventing and treating symptoms of those toxicities, and infections associated with the toxicities. It has been determined that patients are accepting of LED photomodulation treatment, as the treatment is administered painlessly, easily, and generally the process is not overly time-consuming. Very little additional time is required for the patient beyond other therapies, and in many cases the LED photomodulation treatment lasts for less than 10 minutes, and often for less than 1 minute. LED photomodulation treatment therefore increases patient compliance with EGFR inhibitor therapy and thereby increases the success of treatment with EGFR inhibitor agents for different types of cancer.

In some embodiments, the method of the invention is effective for treating, preventing, and preventing the progression of toxicity to the skin, hair, and/or nails, that is associated with the administration of EGFR inhibitors. Skin refers to all layers of the skin, including the epidermis, the dermis, and subcutaneous layer (also known as the hypodermis or subcutis), and includes any structure or portion found within any of these layers, and any structure or portion that may traverse any of these layers, and includes, but is not limited to hair follicles and sebaceous glands.

The method of the invention comprises using LED photomodulation treatment in order to treat a subject in need thereof, either alone or in combination with other agents, to prevent or treat toxicities to the skin, hair, and nails that are associated with EGFR inhibitor treatment. The method comprises directing light onto a target area on said subject, the light being emitted from one or more LED sources that produces at least one range of wavelengths of light.

In one embodiment, a method is provided for preventing or treating inflammation of the skin, hair, and/or nails, associated with administration of one or more EGFR inhibitors in a subject in need thereof.

In one embodiment the LED source used in the method of the invention emits light at a wavelength from about 300 nm to about 1600 nm. In a preferred embodiment, the LED source emits light at a wavelength from about 550 nm to about 650 nm. In another preferred embodiment, the wavelength is about 590 nm.

In another preferred embodiment, a combination of wavelengths is used, the combination comprising about 90% of a wavelength of about 590 nm, and about 10% of a wavelength of about 870 nm.

In some embodiments, the LED source used in the method of the invention emits light in pulses. Pulses may be at various durations and intervals. In one preferred embodiment, pulses are 250 ms in duration and are repeated 100 times, and are separated by 100 ms in a single treatment.

In one preferred embodiment, the fluence for a single treatment is less than about 1.0 J/cm². In another one preferred embodiment, the fluence for a single treatment is from about 0.1 to about 0.9 J/cm². In yet another preferred embodiment, the fluence is about 0.15 J/cm². In yet another preferred embodiment, the fluence is about 0.10 J/cm².

In one embodiment, the LED phototherapy treatment according to the method of the invention is administered once daily. In some embodiments, the LED phototherapy treatment is administered beginning prior to the administration of EGFR inhibitor therapy and continues during EGFR inhibitor therapy. In other embodiments, the LED phototherapy treatment is administered concurrent with the administration of EGFR inhibitor therapy.

In one embodiment, the LED photomodulation treatment according to the method of the invention is administered following the initial dose of EGFR inhibitor therapy. In another embodiment the LED photomodulation treatment according to the method of the invention is administered starting after the final dose of EGFR inhibitor is given to a subject.

In one embodiment, a method is provided for reducing vascular dilatation in the skin, that is associated with the administration of EGFR inhibitors.

In another embodiment, a method is provided for reducing permeability and reducing activation of nociceptive fibers in skin that is associated with the administration of EGFR inhibitors.

In one embodiment, a method is provided for preventing or treating toxicity to the skin, including without limitation, the epidermal, dermal, and/or subcutaneous layer of the skin that is associated with the administration of one or more EGFR inhibitors in a subject in need thereof. The method comprises using light LED phototherapy treatment which comprises directing light onto a target area of the skin of said subject, the light being emitted from one or more LED sources that produces at least one range of wavelengths of light.

In one preferred embodiment, the method of the invention prevents or treats skin toxicity in the form of an acneiform rash that is not caused by bacteria.

In another embodiment, the method of the invention prevents or treats skin toxicity in the form of a papulopustular rash. In another embodiment, the method of the invention prevents or treats skin toxicity in the form of a maculo-papular rash.

In another embodiment, the method of the invention prevents or treats skin toxicity in the form pruritis.

In yet other embodiments, the method of the invention treats skin that is classified as an NCI-CTC grade 1, grade 2, grade 3, or grade 4 rash. In one preferred embodiment, the method of the invention treats skin that is classified as an NCI-CTC grade 2 or higher.

In some embodiments, the method of the invention comprises using LED photomodulation treatment and further comprises the administration of one or more additional agents. In some embodiments, the additional agent is lotion containing copper.

In one embodiment, the skin toxicity to be treated is in an area of the skin selected from the group consisting of the epidermis, the dermis, and the subcutaneous layer of the skin.

In some embodiments, LED photomodulation therapy is directed to one or more target areas, which comprise, but are not limited to the face, neck, chest, forehead, back, scalp, hands, and feet.

In a preferred embodiment, LED photomodulation therapy is directed to the face. In another preferred embodiment, LED photomodulation therapy is directed to the hands and/or feet.

In one embodiment, the EGFR inhibitor is selected from the group consisting of cetuximab, erlotinib, gefitinib, panitumumab, zalutumumab, nimotuzumab, matuzumab, and lapatinib.

### DETAILED DESCRIPTION

The EGFR and its ligands play a critical role in over 70% of all cancers. The enhanced activity of this receptor is a hallmark of many human malignancies, including breast, lung, prostate, thyroid, head and neck, ovary, stomach, kidney, brain, pancreatic, glioblastoma, and renal cell carcinoma, among others. Therefore, drugs targeting the epidermal growth factor system play an important role in the treatment of many different types of cancer.

This invention encompasses methods of treating and preventing toxicity of the skin, hair, and nails, that is associated with the administration of EGFR inhibitors, comprising using light-emitting diode (LED) photomodulation therapy, which is a non-thermal light therapy, either alone or in combination with other therapies. Skin refers to all layers of the skin, including the epidermis, the dermis, and subcutaneous layer (also known as the hypodermis or subcutis), and includes any structure or portion found within any of these layers, and any structure or portion that may traverse any of these layers, and includes, but is not limited to hair follicles and sebaceous glands. Subjects to be treated with this invention are mammalian, and preferably are human. By treating patients in need of treatment using the methods of this invention, one can eliminate, manage, attenuate, ameliorate, or prevent the progression of skin, hair, and nail toxicity in a subject associated with the administration of one or more EGFR inhibitors.

The method of the invention encompasses LED photomodulation treatment for preventing or treating any kind of toxicity to tissues of the body associated with the administration of EGFR inhibitors, and in particular, any area of the skin. The method of the invention also encompasses preventing or treating toxicities to the hair and nails that are associated with administration of one or more EGFR inhibitors.

The method of preventing and treating the multiple forms of skin, hair, and nail toxicities associated with EGFR inhibitor treatment is accomplished according to the invention by treating a subject in need of treatment with LED photomodulation at the affected area in need of treatment. In the method according to the invention, light from at least one LED source is directed to one or more targeted areas of a subject's skin, hair, and/or nails, for a specified duration, at a specified wavelength or range of wavelengths, in an either pulsed or continuous fashion. Treatment may begin prior to, during, or following initiation of EGFR inhibitor treatment, and can last for various amounts of time.

Any source or sources of LED known to one of ordinary skill in the art may be utilized in the methods of the invention. It is preferred that the panel which emits the light allows for uniform administration of light therapy. The LEDs may be assembled into small lamps, for example, up to about 3 mm to about 5 mm in diameter, but about 10 mm and larger lamps may also be used. LEDs may also be assembled into larger arrays or panels, which allow for higher energy intensities. Large LED panel arrays can also allow larger areas to be treated at one time, such as the entire face. For example, the LEDs may be assembled into lamps of between about 70 mm to about 100 mm inches mm in diameter. In a preferred embodiment, the LEDs are about 80 mm in diameter. The LED arrays may be arranged in such a way to reach the desired target areas on the subject, such that, for example, the contours on the face do not prevent any areas from being reached by the light.

Any source of low level light may be used, such that it emits, preferably, less than 1 J/cm². In one preferred embodiment, the device used for emitting light is Gentlewaves® (LightBioScience, LLC, Virginia Beach, Va.).

An LED or an array of LEDs can be used to emit light at one or more wavelengths, either simultaneously or consecutively, to deliver energy fluence to the targeted area or areas on the subject. The targeted cells are provided with a clinically effective fluence of energy to initiate photomodulation and/or photoregeneration, but do not receive an amount of light that could cause damage to the cells that are targeted.

In some embodiments, the array of LEDs can be used to deliver a continuous wave of light to the targeted area. Alternatively, and in a preferred embodiment, the light source may be “pulsed” according to a pattern determined to be effective depending on the nature of the targeted area and the actual or anticipated severity of symptoms. The pattern, for example, may be referred to by the duration of each pulse, the time between each pulse, and the number of pulses administered. A pattern of “250/100/100,” for example, would refer to pulses of 250 milliseconds in duration, separated by 100 milliseconds, and repeated 100 times. Such a pattern may deliver the same energy fluence as a 25 second continuous wave treatment.

In one preferred embodiment, the pulse pattern is 250/100/100.

The LED array may include LED emitters that emit multiple wavelengths, a single wavelength, or the array may include multiple types of emitters, if more than one wavelength is used for treatment. Each LED will generally emit at a dominant emissive wavelength from about 300 nm to about 1600 nm. The array may include combinations of LEDs that emit in the visible and/or infrared portion of the spectrum.

Wavelength is chosen based on the particular target area to be treated and on the severity of the symptoms or anticipated symptoms to be treated or prevented, as well as on the desired effect. The wavelength or wavelengths must reach the cells of the target area to be effective, and the tissue penetration depth required may differ depending on, for example, the nature of the target area and the particular condition to be prevented or treated. For example, in most cases, the wavelength used for damaged skin is likely to be different from the wavelength used for non-damaged skin.

In one embodiment, the LED emits a single wavelength from about 300 nm to about 1600 nm. In one embodiment, the LED emits a single wavelength from about 300 nm to about 400 nm. In one embodiment, the LED emits a single wavelength from about 400 nm to about 500 nm. In one embodiment, the LED emits a single wavelength from about 500 nm to about 600 nm. In one embodiment, the LED emits a single wavelength from about 600 nm to about 700 nm. In one embodiment, the LED emits a single wavelength from about 700 nm to about 800 nm. In one embodiment, the LED emits a single wavelength from about 800 nm to about 900 nm. In one embodiment, the LED emits a single wavelength from about 900 nm to about 1000 nm. In one embodiment, the LED emits a single wavelength from about 1000 nm to about 1100 nm. In one embodiment, the LED emits a single wavelength from about 1100 nm to about 1200 nm. In one embodiment, the LED emits a single wavelength from about 1200 nm to about 1300 nm. In one embodiment, the LED emits a single wavelength from about 1300 nm to about 1400 nm. In one embodiment, the LED emits a single wavelength from about 1400 nm to about 1500 nm. In one embodiment, the LED emits a single wavelength from about 1500 nm to about 1600 nm.

In one preferred embodiment, the LED emits a single wavelength from about 400 nm to about 800 nm. In another preferred embodiment, the LED emits a single wavelength from about 500 nm to about 700 nm.

In yet another preferred embodiment, the LED emits a single wavelength from about 500 nm to about 650 nm.

In yet another preferred embodiment, the LED emits a single wavelength of about 590 nm.

In some preferred embodiments, combinations of light in the visible spectrum and light in the infrared range are emitted by the LED source or sources. In one preferred embodiment, a combination is used of visible wavelength such as yellow, from about 570 nm to about 610 nm, and infrared wavelength, from about 900 nm to about 1000 nm.

In another preferred embodiment, the combination of light comprises about 90% of a wavelength of about 590 nm, and about 10% of a wavelength of about 870 nm.

Pulse duration is determined based on the particular target area to be treated, and on the severity of the symptoms or anticipated symptoms to be treated or prevented, as well as on the desired effect. Pulse duration refers to the time over which the target area is exposed to the LED during each pulse, and in some embodiments is from about 0.1 microseconds to about 1 hour. In one embodiment, the pulse duration is from about 1.0 millisecond to about 1 hour. In another embodiment, the pulse duration is from about 10 milliseconds to about 1 hour. In another embodiment, the pulse duration is from about 20 milliseconds to about 1 hour. In another embodiment, the pulse duration is from about 50 milliseconds to about 1 hour. In another embodiment, the pulse duration is from about 100 milliseconds to about 1 hour. In another embodiment, the pulse duration is from about 150 milliseconds to about 1 hour. In another embodiment, the pulse duration is from about 200 milliseconds to about 1 hour. In another embodiment, the pulse duration is from about 250 milliseconds to about 1 hour. In another embodiment, the pulse duration is from about 300 milliseconds to about 1 hour. In another embodiment, the pulse duration is from about 400 milliseconds to about 1 hour. In another embodiment, the pulse duration is from about 500 milliseconds to about 1 hour. In another embodiment, the pulse duration is from about 1 second to about 1 hour.

In one preferred embodiment, the pulse duration is from about 100 milliseconds to about 800 milliseconds. In another preferred embodiment, the pulse duration is from about 100 milliseconds to about 500 milliseconds.

In yet another preferred embodiment, the pulse duration is from about 1 second to about one minute.

In yet another preferred embodiment, the pulse duration is about 250 milliseconds.

In some embodiments, it is more desirable to deliver a continuous wave of light to the targeted area rather than pulsed light, depending on the nature of the targeted area and the actual or anticipated severity of symptoms.

If pulsed light is delivered to the target area, then pulse frequency may be from about 2 to about 10,000 pulses per treatment. In some embodiments, the pulse frequency is from about 10 to about 1,000 pulses per treatment. In other embodiments, the pulse frequency is from about 50 to about 500 pulses per treatment.

In one preferred embodiment, the pulse frequency is from about 75 to about 200 pulses per treatment. In another preferred embodiment, the pulse frequency is about 100 pulses per treatment.

The interval in between pulses is, in one embodiment, from about 0.1 milliseconds to about 1 minute. In another embodiment, the interval in between pulses is from about 0.5 milliseconds to about 30 seconds. In another embodiment, the interval in between pulses is from about 1.0 millisecond to about 10 seconds. In another embodiment, the interval in between pulses is from about 50 milliseconds to about 10 seconds. In a preferred embodiment, the interval in between pulses is from about 75 milliseconds to about 1 second. In a preferred embodiment, the interval in between pulses is between about 100 milliseconds and about 300 milliseconds. In another preferred embodiment, the interval in between pulses is about 100 milliseconds.

The total energy fluence delivered in a single treatment varies based on the specific targeted area or areas being treated and the severity of the symptoms or anticipated symptoms, but will generally be less than about 10 J/cm² in order to prevent possible side effects. When the light is administered indirectly to the target area, the fluence at the source may be much higher than 10 J/cm², but the fluence perceived by the source may be very low, due to the absorption and scattering of the light by tissue, bone, or other structures between the light source and the targeted cells. In some cases, a fluence reaching the targeted area may be as low as a few nanojoules.

In a preferred embodiment, the fluence for a single treatment is less than about 1.0 J/cm². In one preferred embodiment, the fluence for a single treatment is from about 0.1 to about 0.9 J/cm². In another preferred embodiment, the fluence is about 0.15 J/cm². In yet another preferred embodiment, the fluence is about 0.10 J/cm².

In one preferred embodiment, the LED treatment comprises administering light at 590 nm, with a pulse duration of 250 milliseconds, a pulse frequency of 100, with 100 milliseconds in between pulses, at a fluence of 0.15 J/cm².

It can be advantageous to begin LED treatment prior to the appearance of toxicity to the skin, hair, and nails in order to prevent or treat toxicity associated with EGFR inhibitor treatment. In some embodiments, LED photomodulation treatment begins prior to the administration of EGFR inhibitors. In some embodiments, LED photomodulation treatment begins about 8 weeks, about 7 weeks, about 6 weeks, about 5 weeks, about 4 weeks, about 3 weeks, about 2 weeks, or about 1 week prior to EGFR inhibitor administration. In other embodiments, LED photomodulation treatment begins from about 1 to about 2 weeks prior to EGFR inhibitor administration.

In a preferred embodiment, LED photomodulation treatment begins from about 1 to about 7 days prior to EGFR inhibitor administration. In another preferred embodiment, LED photomodulation treatment begins from about 3 to about 5 days prior to EGFR inhibitor administration.

In other embodiments, LED photomodulation treatment begins following the appearance of toxicity to the skin, hair, and/or nails. In other embodiments, LED photomodulation treatment begins prior to the appearance of toxicity, but following the subject's described discomfort to the skin, hair, and/or nails.

LED photomodulation treatment may be administered daily or at various intervals. Accordingly, LED photomodulation treatment may also be administered every other day, or every two days. In other embodiments, LED photomodulation treatment may be administered once per week, 2 times per week, 3 times per week, 4 times per week or 5 times per week.

In some embodiments, on days on which LED photomodulation treatment is administered, LED photomodulation treatment is administered once per day, twice per day, 3 times per day, or 4 times per day. In other embodiments LED photomodulation treatment is administered more than 4 times per day, depending on the desired effect of the treatment and the severity of the toxicity to be treated.

In some embodiments, when at least some of the LED photomodulation treatment is administered to a subject on the same day as the administration of EGFR inhibitor, the LED photomodulation treatment is administered prior to the administration of EGFR inhibitor. In other embodiments, when at least some of the LED photomodulation treatment is administered on the same day as the administration of EGFR inhibitor, the LED photomodulation treatment is administered following the administration of EGFR inhibitor.

In some embodiments, the total duration of LED photomodulation treatment a subject receives in a day is about 1 hour or less, and in other embodiments is about 30 minutes or less. In other embodiments, the total duration of LED photomodulation a subject receives in a day is about 25 minutes or less, about 20 minutes or less, about 15 minutes or less, or about 10 minutes or less. In a preferred embodiment, the total duration of LED photomodulation treatment a subject receives in a day is about 5 minutes or less. In another preferred embodiment, the total duration of LED photomodulation treatment a subject receives in a day is about 1 minute or less. In another preferred embodiment, the total duration of LED photomodulation treatment a subject receives in a day is about 30 seconds or less.

In some embodiments, LED photomodulation treatment is administered to a subject until the final dose of EGFR inhibitor is administered. In other embodiments, LED photomodulation treatment continues following the final dose of EGFR inhibitor, or for as long as it continues to exert a beneficial effect on the area or areas being treated. In some embodiments, LED photomodulation treatment continues from about one week to about 16 weeks following the final dose of EGFR inhibitor. In some embodiments, LED photomodulation treatment continues from about one week to about 12 weeks or more, following the final dose of EGFR inhibitor. In some embodiments, LED photomodulation treatment continues from about one week to about 8 weeks following the final dose of EGFR inhibitor. In other embodiments, LED photomodulation treatment continues from about one week to about 4 weeks following the final dose of EGFR inhibitor.

In some embodiments, LED photomodulation treatment is initiated following the final dose of EGFR inhibitor. Depending on the duration of LED photomodulation treatment, the frequency with which LED photomodulation treatment is administered may change over time. In some embodiments, LED photomodulation treatment continues from about one week to about 8 weeks following the final dose of EGFR inhibitor. In other embodiments, LED photomodulation treatment continues from about one week to about 8 weeks following the final dose of EGFR inhibitor. In a preferred embodiment, LED photomodulation treatment continues for about 4 weeks following the final dose of EGFR inhibitor. In another preferred embodiment, LED photomodulation treatment continues for about 30 days following the final dose of EGFR inhibitor. In another preferred embodiment, LED photomodulation treatment continues from about 10 days to about 90 days following the final dose of EGFR inhibitor. In another preferred embodiment, LED photomodulation treatment continues from about 10 days to about 60 days following the final dose of EGFR inhibitor.

In some embodiments, LED photomodulation treatment is continued until the toxicity to be treated or its symptoms have improved, or until symptoms are no longer present or until the toxicity has been eliminated. In some embodiments, the toxicity or symptoms to be ameliorated, prevented, or treated include but are not limited to one or more of a subject's discomfort, pain, itching, sensitivity to touch, swelling, discoloration, burning, or change in hair amount, texture, or pattern on the head, eyelashes, eyebrows, or elsewhere on the body.

In some embodiments, the LED photomodulation treatment is administered in order to prevent or treat one or more of a subject's discomfort, pain, itching, sensitivity to touch, swelling, discoloration, or burning associated with the administration of one or more EGFR inhibitors.

In some embodiments, LED photomodulation treatment begins following the final dose of EGFR inhibitor, and continues from about one day to about 8 weeks, or longer. In other embodiments, LED photomodulation treatment begins following the final dose of EGFR inhibitor, and continues until symptoms have improved or until symptoms are no longer present, or while the LED photomodulation treatment continues to have a beneficial effect on the area or areas treated.

The method of the invention comprising LED photomodulation treatment for the prevention or treatment of toxicity to the skin, hair and/or nails may be used in combination with other treatments or agents for toxicities to the skin, hair and/or nails. These additional treatments or agents may aid in treating the toxicity or may alleviate or eliminate the symptoms associated with the toxicity. The additional treatments or agents may also aid in the effectiveness of the LED therapy. In some embodiments, LED photomodulation treatment is used in combination with one or more agents, which include but are not limited to skin moisturizers; lotions; sunscreens; topical anti-inflammatory agents; topical steroids; oral steroids; topical antibiotics; oral antibiotics; topical cleansers; white vinegar soaks; aluminum soaks; Burrow's solution; Monsel's solution; silver nitrate; thymol, emolliants such as Bag Balm and Petroleum jelly; mild soap; solutions of ammonium lactate, salicylic acid and urea; protective coverings; zinc oxide cream; liquid cyanoacrylate preparations; warm compresses; analgesics; tacrolimus cream; artificial tears; and antifungal agents.

Oral or topical antibiotics may, for example, include tetracylcine, minocycline, doxycyline, polymyxin B, Clindamycin, and Neomycin.

Topical steroids may include, for example, hydrocortisone cream and dexamethasone ointment.

Sunscreen may include, for example, that which is PABA free, preferably with UVA/UVB protection. In some embodiments, the sunscreen has an SPF of ≧15. Use of sunscreens with higher SPF values may require use of LED treatments that deliver higher energy fluence.

In one preferred embodiment, the agents administered in combination with LED photomodulation treatment, whether administered prior to LED treatment, simultaneously with LED treatment, extending, optionally beyond LED treatment, or which are administered following LED treatment, are lotion products containing copper.

The EGFR inhibitors contemplated for use according to the methods of the present invention can be any drug which acts as an inhibitor of the EGF receptor, including small molecules, antibodies, or any other class of agents. Such inhibitors include those already known to those of skill in the art, but may include inhibitors subsequently developed. EGFR inhibitors include, but are not limited to gefitinib (Iressa®), erlotinib (Tarceva®), cetuximab (Erbitux®), panitumumab (Vectibix®,) imatinib (Gleevec®), zalutumumab, nimotuzumab, matuzumab, and lapatinib.

Toxicity as used herein refers to any untoward reactions to the administration of any one or more EGFR inhibitors.

This invention encompasses preventing and treating toxicity of the external surface of the body, including the skin, hair, and/or nails, wherein the toxicity is associated with the administration of one or more EGFR inhibitors. The invention also encompasses preventing and treating toxicity to any part of the skin which is not on the external surface of the body, such as the dermal and subcutaneous layers of the skin, and any structure or portion found within any of these layers, and any structure or portion that may traverse any of these layers, including, but not limited to hair follicles and sebaceous glands. The treatment according to the invention comprises using LED photomodulation treatment in patients for which one or more EGFR inhibitors might be indicated, such patients with cancer, for example. In addition, the invention embodies preventing or treating toxicity of the skin, hair, and nails associated with the administration of one or more EGFR inhibitors with LED photomodulation treatment in patients administered one or more EGFR inhibitors for any indication other than cancer for which one or more EGFR inhibitors might be indicated.

The method of the invention contemplates treating both short-term and long-term toxicities of the skin, hair, and nails associated with the administration of one or more EGFR inhibitors. Short-term toxicities comprise those which improve within about 3 months following the discontinuation of treatment with EGFR inhibitors. Long-term toxicities comprise those which do not improve within about 3 months following the discontinuation of treatment with EGFR inhibitors. Most short-term toxicities resolve within about 1 to about 3 months.

In one embodiment, LED photomodulation is administered to patients to prevent or treat conditions of the skin associated with EGFR inhibitors, and in particular to prevent or treat rashes of the skin associated with EGFR inhibitors. While acne is a rash caused by propionibacteria, the acneiform rashes associated with EGFR inhibitors, that are prevented or treated by the method of the invention are not a result of bacteria. In one embodiment, the invention prevents or treats rashes that comprise acneiform rashes that are not associated with bacteria. The method of the invention encompasses preventing or treating, in some preferred embodiments, a papulopustular rash associated with the administration of one or more EGFR inhibitors. In another embodiment, the method of the invention prevents or treats a maculo-papular rash associated with the administration of one or more EGFR inhibitors. In another embodiment of the invention, dermatitis associated with the administration of one or more EGFR inhibitors may be prevented or treated. In yet another embodiment, the invention encompasses preventing or treating a morbilliform rash associated with the administration of one or more EGFR inhibitors.

The method of the invention also prevents or treats toxicity of the skin, hair, and nails following the discontinuation of treatment with EGFR inhibitors.

In certain embodiments, the method of the invention encompasses preventing or treating one or more of the following, which are associated with the administration of one or more EGFR inhibitors: psoriasis, hypopigmentation, hyperpigmentation, fissures, pruritis, xerosis, and telangiectasias.

The method of the invention also comprises preventing or treating toxicities to the nails that are associated with the administration of EGFR inhibitors. In certain embodiments, the method of the invention encompasses preventing or treating paronychia associated with the administration of one or more EGFR inhibitors. The method of the invention further contemplates preventing or treating secondary infections of the nail beds that are associated with the administration of one or more EGFR inhibitors.

The methods of the invention also comprise preventing or treating toxicities of the eyelids that are associated with the administration of EGFR inhibitors, including, but not limited to blepharitis, ectropion and entropion.

The method of the invention further comprises treating or preventing disturbances to the normal hair growth cycle that are associated with administration of EGFR inhibitors. In one embodiment, the method of the invention encompasses preventing or treating hair loss or alopecia associated with the administration of one or more EGFR inhibitors. In yet other embodiments, the invention encompasses preventing or treating increases in the amount of and/or texture of facial hair associated with administration of one or more EGFR inhibitors, and preferably, in women. In yet other embodiments, the method of the invention encompasses preventing or treating changes in the texture or amount of hair on the head or on the eyebrows, that are associated with the administration of one or more EGFR inhibitors.

Administration of EGFR inhibitors is also associated with alterations in the texture, length, and direction of growth of eyelashes. In one embodiment, the method of the invention encompasses preventing or treating trichomegaly and/or hypertrichosis associated with the administration of one or more EGFR inhibitors.

The National Cancer Institute (NCI) classifies rashes according to the NCI Common Toxicity Criteria (NCI-CTC), and includes categories that range from grade 1 to grade 4.

Grade 1 comprises macular or papular eruption or erythema with or without associated symptoms. Grade 2 comprises macular or papular eruption, or erythema with pruritus or associated symptoms covering less than 50% of the body surface or localized desquamation or other lesions covering less than 50% of the body surface. Grade 3 comprises symptomatic, generalized erythroderma, maculopapular, vesicular eruption or desquamation covering greater than or equal to 50% of the body surface. Grade 4 comprises generalized exfoliative dermatitis, ulcerative dermatitis, or bullous dermatitis.

This invention contemplates preventing or treating any of NCI-CTC grade 1 to grade 4 rashes, including any rashes that might be in between stages or that can be described by more than one stage or by other means of classification.

In some embodiments, the method of the invention is used to prevent or treat NCI-CTC-grade 1 rashes, grade 2 rashes, grade 3 rashes, or grade 4 rashes associated with EGFR inhibitor treatment.

The method of the invention also encompasses treating any areas of skin that are affected by the administration of one or more EGFR inhibitors. In some embodiments, the method of the invention is used to prevent or treat toxicity to the skin present on one or more of the face, forehead, chest, back, neck, arms, legs, shoulders, hands, feet, fingers, toes, or scalp, or any other area of skin on a human which may be affected by treatment with EGFR inhibitors. The method of the invention also encompasses treating any areas of toenails or fingernails that are affected by the administration of one or more EGFR inhibitors, as well as growth of toenails or fingernails.

In a preferred embodiment, the method of the invention is used to prevent or treat skin toxicities on the face or forehead associated with EGFR inhibitor treatment. In another preferred embodiment, the method of the invention is used to prevent or treat skin toxicities on the chest or back. In yet another preferred embodiment, the invention is used to prevent or treat toxicities on the hands or feet.

In other embodiments, the invention encompasses preventing or treating blisters and erythema of the hands and feet, which are often under pressure due to walking and other activity, and which EGFR inhibitors make susceptible to such injury due to damage that EGFR inhibitors cause to the capillary endothelia.

Infectious complications of the skin, hair, and/or nails may occur from EGFR inhibitor administration. The invention also encompass preventing or treating any kind of infections in any areas of skin, hair, or nails that are affected by the administration of one or more EGFR inhibitors. These infections may be, but are not limited to, bacterial infections such as Impetigo or Dissecting cellulitis, viral infections, and fungal infections.

The method of the invention encompasses preventing or treating inflammation of any areas of skin, hair, or nails that is associated with the administration of one or more EGFR inhibitors.

In a preferred embodiment, the method of the invention prevents or treats an inflammatory rash on any one or more areas of the skin, associated with administration of one or more EGFR inhibitors.

In one embodiment, a method is provided for reducing vascular dilatation in the skin, that is associated with the administration of EGFR inhibitors.

In another embodiment, a method is provided for reducing permeability and reducing activation of nociceptive fibers in skin, skin, that is associated with the administration of EGFR inhibitors.

In another embodiment, the invention provides for improved wound healing on the skin of a subject, by using LED photomodulation therapy.

The method of the invention also comprises preventing or treating toxicities of the skin, hair, or nails that are associated with the administration of EGFR inhibitors and one or more additional agents that are administered to a subject as part of cancer treatment, or as part of the treatment for any other disorder for which EGFR inhibitors are indicated, either concurrently or within one or a plurality of days of administration of an EGFR inhibitor. The additional agent(s) administered to a subject as part of cancer treatment or other treatment might exacerbate the toxicity associated with the one or more EGFR inhibitors.

### EXAMPLES

It is understood that the following examples and embodiments described herein are for illustrative purposes only and that various modifications or changes in light thereof will be suggestive to persons skilled in the art and are to be included within the spirit and purview of this application and the scope of the appended claims. All publications, patents, and patent applications cited herein are hereby incorporated by reference in their entirety for all purposes.

### Example 1

### LED Treatment Improves Dermatologic Toxicities Associated with EGFR Inhibitors

Three cancer patients were assessed who had been administered EGFR inhibitor therapy. The patients each had a papulopustular rash which was present mostly on the face and hands, and which was associated with EGFR inhibitor therapy. The rashes on each patient were classified as severe in nature both by appearance and by the self-reporting of the patients. The rashes appeared as thermal or chemical burns to the skin.

LED photomodulation treatment was administered to the three patients following the appearance of the severe rash. The Gentle Waves® LED device (LightBioScience, LLC, Virginia Beach, Va.) was used to administer the light. LED treatments were administered at a preset cycle, 590 nm, standard 100-pulse, 250 milliseconds per pulse at a fluence of 0.15 J/cm². LED phototherapy was administered to each patient daily for two weeks. In addition to daily LED phototherapy treatment, the patients applied lotion products containing copper to areas of the rash.

All three patients responded well and quickly to the treatment. After the first three treatments, the patients noted improvement in how their skin felt. After the first week of treatment, visible signs of healing were noted, in that the rashes appeared less severe.

Certain modifications and improvements will occur to those skilled in the art upon a reading of the foregoing description. It should be understood that all such modifications and improvements have been deleted herein for the sake of conciseness and readability but are properly within the scope of the following claims.

1. A method of treating or preventing skin toxicity associated with administration of an epidermal growth factor receptor (EGFR) inhibitor in a subject in need thereof, said method, comprising directing light onto a target area on said subject, said light being emitted from one or light emitting diode (LED) sources producing at least one range of wavelengths of light.

2. The method according to claim 1, wherein said toxicity is associated with inflammation of said skin.

3. The method according to claim 1, wherein said EGFR inhibitor is selected from the group consisting of cetuximab, erlotinib, gefitinib, and panitumumab.

4. The method according to claim 1, wherein said skin toxicity is in the epidermis.

5. The method according to claim 1, wherein said skin toxicity is in the dermis.

6. The method according to claim 1, wherein said skin toxicity is in the subcutaneous layer of the skin.

7. The method according to claim 1, wherein said skin toxicity is an acneiform rash that is not caused by bacteria.

8. The method according to claim 1, wherein said skin toxicity is a papulopustular rash.

9. The method according to claim 1, wherein said skin toxicity is pruritis.

10. The method according to claim 1, wherein said skin toxicity is classified as an NCI-CTC grade 1, grade 2, grade 3, or grade 4 rash.

11. The method according to claim 10, wherein said skin toxicity is classified as an NCI-CTC grade 2.

12. The method according to claim 1, further comprising administration of one or more additional agents.

13. The method according to claim 12, where in the agent is lotion containing copper.

14. The method according to claim 1, wherein the target area is selected from the group consisting of the face, neck, back, scalp, hands, and feet.

15. The method according to claim 1, wherein the LED source emits light at a wavelength from about 500 nm and about 700 nm.

16. The method according to claim 1, wherein the LED source emits light at a wavelength of about 590 nm.

17. The method according to claim 1, wherein the LED source emits light in pulses that are 250 ms in duration that are separated by 100 ms, and that is repeated 100 times.

18. The method according to claim 1, wherein the light from the LED source is administered once daily.

19. The method according to claim 1, wherein the light from the LED source is administered beginning prior to the administration of EGFR inhibitor therapy.

20. The method according to claim 1, wherein the light from the LED source is administered concurrent with the administration of EGFR inhibitor therapy.

21. The method according to claim 1, wherein the light from the LED source is administered following the initial dose of EGFR inhibitor therapy.

22. The method according to claim 1, wherein the LED delivers a total energy fluence of 0.15 J/cm².
